(19) United States
(12) Patent Application Publication
(10) Pub. No.: US 2022/0265460 A1
(43) Pub. Date: Aug. 25, 2022

(54) RESERVOIR-LESS EXTERNAL CATHETER

(71) Applicant: Charles Coker, Yantis, TX (US)

(72) Inventor: Charles Coker, Yantis, TX (US)

(21) Appl. No.: 17/180,869

(22) Filed: Feb. 22, 2021

(51) Int. Cl. A61F 5/453; A61F 5/455; A61F 5/44

(52) U.S. Cl. CPC A61F 5/453; A61F 5/455; A61F 5/4405

(57) ABSTRACT

Illustrated views of a first exemplary external catheter and a second external catheter for providing medical help to relieve a bladder of a person are presented. The first external catheter is intended for male subjects while the second external catheter is intended for female subjects, though each a male or a female may use either of the first external catheter and/or the second external catheter. The first external catheter and the second external catheter are useful for a person to urinate to remove toxins or waste from their body. The first external catheter and the second external catheter provide a safe and clean environment, thus reducing disease or infections.

### FIELD OF THE INVENTION

This invention relates to catheters. More particularly, it relates to reservoir-less catheters.

### BACKGROUND

In medicine, a catheter is a thin tube made from medical grade materials serving a broad range of functions. Catheters are medical devices that can be inserted in the body to treat diseases or perform a surgical procedure. By modifying the material or adjusting the way catheters are manufactured, it is possible to tailor catheters for cardiovascular, urological, gastrointestinal, neurovascular, and ophthalmic applications.

Catheters can be inserted into a body cavity, duct, or vessel. Functionally, they allow drainage, administration of fluids or gases, access by surgical instruments, and also perform a wide variety of other tasks depending on the type of catheter. The process of inserting a catheter is “catheterization”. In most uses, a catheter is a thin, flexible tube (“soft” catheter) though catheters are available in varying levels of stiffness depending on the application. A catheter left inside the body, either temporarily or permanently, may be referred to as an “indwelling catheter” (for example, a peripherally inserted central catheter). A permanently inserted catheter may be referred to as a “permcath” (originally a trademark).

There are two general types of catheters, inserted catheters and external catheters. External catheters, generally, are used to help alleviate a person of waste through the urinary track. These catheters include, but are not limited to, hydrophilic (self-lubricated) catheters, intermittent straight catheters, pediatric catheters, etc. Each of the external catheters have one or more reservoirs. The urine is collected and stored for disposal within one of the reservoirs of the external catheters. This can cause issues in storing or disposal of the external catheters. This can cause a person to feel less than they are by having others see or to smell the catheter. Further, the earlier the disposal of the urine, the safer it is for those surrounding and caring for the person.

Accordingly, and in light of the foregoing, there is a need for a device that can collect urine from an individual without a reservoir.

### BRIEF DESCRIPTION OF THE DRAWINGS

FIG. 1 is an illustrated view of an exemplary external catheter for males.

FIG. 2 is an illustrated view of an exemplary external catheter for females.

### DETAILED DESCRIPTION

The phrases “in one embodiment,” “in various embodiments,” “in some embodiments,” and the like are used repeatedly. Such phrases do not necessarily refer to the same embodiment. The terms “comprising,” “having,” and “including” are synonymous, unless the context dictates otherwise. Such terms do not generally signify a closed list.

“Above,” “adhesive,” “affixing,” “any,” “around,” “both,” “bottom,” “by,” “comprising,” “consistent,” “customized,” “enclosing,” “friction,” “in,” “labeled,” “lower,” “magnetic,” “marked,” “new,” “nominal,” “not,” “of,” “other,” “outside,” “outwardly,” “particular,” “permanently,” “preventing,” “raised,” “respectively,” “reversibly,” “round,” “square,” “substantial,” “supporting,” “surrounded,” “surrounding,” “threaded,” “to,” “top,” “using,” “wherein,” “with,” or other such descriptors herein are used in their normal yes-or-no sense, not as terms of degree, unless context dictates otherwise.

Reference is now made in detail to the description of the embodiments as illustrated in the drawings. While embodiments are described in connection with the drawings and related descriptions, there is no intent to limit the scope to the embodiments disclosed herein. On the contrary, the intent is to cover all alternatives, modifications and equivalents. In alternate embodiments, additional devices, or combinations of illustrated devices, may be added to, or combined, without limiting the scope to the embodiments disclosed herein.

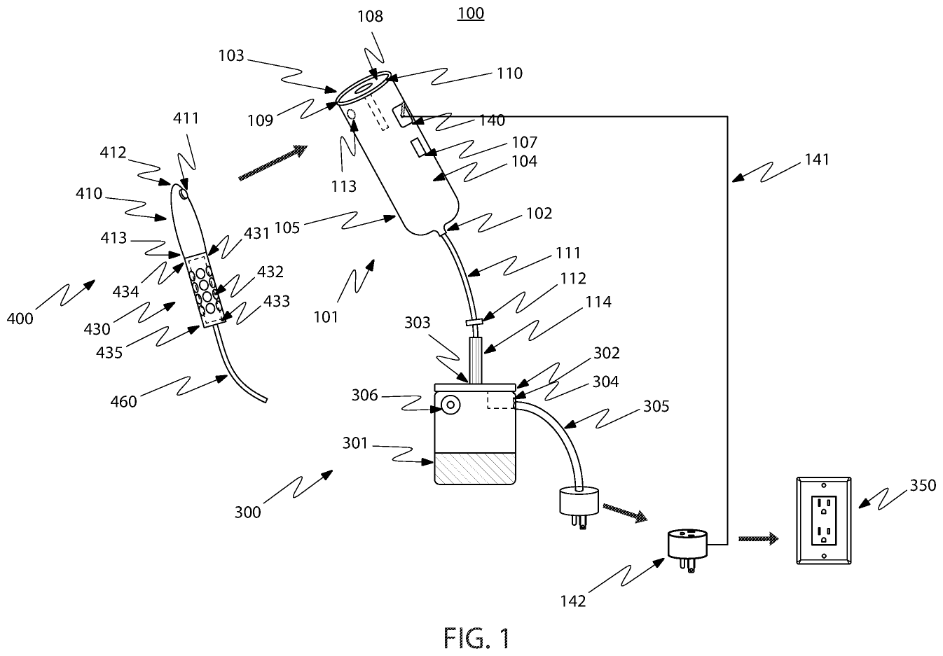

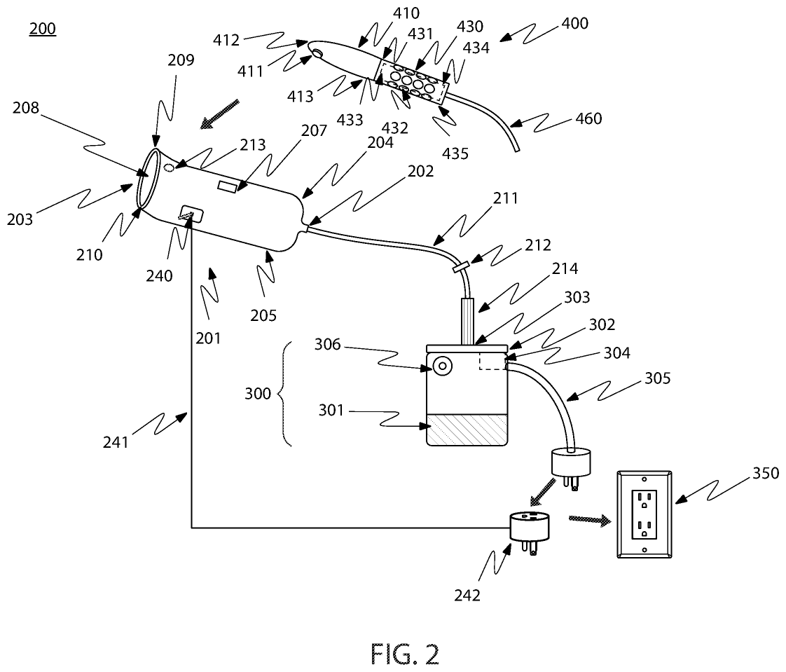

Referring to FIG. 1 and FIG. 2, illustrated views of a first exemplary external catheter 100 and a second external catheter 200 for providing medical help to relieve a bladder of a person is presented. The first extended catheter 100 is significantly similar to the second external catheter 200, where differences may occur will be so drawn to the appropriate first external catheter 100 or the second external catheter 200 as necessary. The first external catheter 100 and the second external catheter 200 may be referenced separately when citing differences, however for features similar in both the first external catheter 100 and the second external catheter 200, the first external catheter 100 and the second external catheter 200 shall be referenced as external catheters 100, 200.

The first external catheter 100 is intended for male subjects while the second external catheter 200 is intended for female subjects, though each a male or a female may use either of the first external catheter 100 and/or the second external catheter 200. The first external catheter 100 and the second external catheter 200 are useful for a person to urinate to remove toxins or waste from their body. The first external catheter 100 and the second external catheter 200 provide a safe and clean environment thus reducing disease or infections from used other types of catheters. The first external catheter 100 and the second external catheter 200 may be used in almost any situation or location, such as a car, public restroom, home, office, etc., without others knowing of the use and without an odor or sight of the catheter 100, 200.

The external catheters 100, 200 have a body 101, 201, and a drain 102, 202. The body 101, 201 has a receiving portion 103, 203, a air intake orifice 113, 213, an inside 104, 204 and an outside 105, 205. Optionally and/or additionally, the body 101, 201 has a first switch 107, 207. The first switch 107, 207 is preferably a squeeze type switch, however other types of switches are hereby contemplated, including, but not limited to, toggle switch, push button switch, toggle switch, etc. The air intake orifice 113, 213 is useful for providing an air flow.

The body 101, 201 is preferably a diameter of three inches (3″), however other diameters are hereby contemplated, including, but not limited to, two and one-half inches (2.5″), three and one-half inches (3.5″), etc. The body is preferably a length of ten to twelve inches (10″-12″)m however other lengths are hereby contemplated, including, but not limited to, nine inches (9″), thirteen inches (13″), etc.

The receiving portion 103 of the first external catheter 100 has an opening 108 and an outer rim 109. The opening 108 is preferably two (2) inches in diameter, however other diameters are hereby contemplated, including, but not limited to, one and one-half inch (1.5″), two and one-half inch (2.5″), etc. The outer rim 109 has a ridge 110. The ridge 110 of the outer rim 109 is intended to prevent a user's underwear from contact with the urine or the receiving port 103. The ridge 110 is preferably one-eighth (⅛) inch in width however other widths are hereby contemplated, including, but not limited to, one-fourth (¼) inch, one-sixteenth ( 1/16) inch, etc.

The receiving portion 203 of the second external catheter 200 has an opening 208 and an outer rim, the opening 208 for allowing access to the inside 204 of the second external catheter 200. The opening 208 has a ridge 210, the ridge 210 being for preventing the user's underwear from being in contact with the second external catheter 200 or urine.

A pipe 111, 211 is coupled to the drain 102, 202 of the external catheters 100, 200. The pipe 111, 211 provides a flow chamber for the waste product from the user to a holding tank 300. The pipe 111, 211 has a valve 112, 212. The valve 112, 212 is preferably a solenoid valve. The valve 112, 212 is configured to allow the flow of waste product or to prevent the flow of the waste product.

An air intake device 400 is removably coupled to the external catheters 100, 200. The air intake device 400 provides an air flow and filtering of the air. Thus, it allows for the prevention of the air intake orifice 113, 213 of the body 101, 201 of the external catheters 100, 200 from being clogged or stopped up for debris, dirt, solidifying of liquid waste, etc.

The air-intake device 400 has a body 410, a filter portion 430 and a vacuum line 460. The body 410 of the air-intake device 400 has an air opening 411 substantially near a top portion 412 of the body 410 of the air-intake device 400. The air opening 411 allows the air to flow through the body 101, 201 of the external catheters 100, 200 by aligning and removably coupling the air opening 411 of the body 410 of the air-intake device 400 to the air intake orifice 113, 213 of the body 101, 201 of the external catheters 100, 200.

A first end 431 of the filter portion 430 of the air-intake device 400 is coupled to a bottom portion 413 of the body 410 of the air intake device. The filter portion 430 of the air-intake device 400 has a plurality of openings 432 and an interior 433. The interior 433 of the filter portion 430 optionally, and or additionally, has a filter 434. The air flows through the filter portion and captures particles that may clog the air intake orifice 113, 213 of the external catheters 100, 200.

The vac line 460 is coupled to a bottom end 435 of the filter portion 430 of the air-intake device 400. The vac line 460 is coupled to the holding tank 300.

The holding tank 300 has a tank 301, a top 302, a port 303, a motor 304 and a power source coupler 305. The pipe 111, 211 is configured to couple to the port 303 of the top 302 of the holding tank 300 to allow the waste product to flow from the external catheters 100, 200 to be held in the tank 301.

The motor 304 is coupled to the top 302 of the holding tank 300. The motor 304 provides a vacuum to pull the waste product from the external catheters 100, 200 into the tank 301 of the holding tank 300. The motor 304 is electrically powered by a power source 350 such as AC, DC, etc.

The motor 304 is coupled to the power source coupler 305 for access to the power source 350. The holding tank 300 further has an on/off switch 306. The on/off switch 306 is useful for allowing electrical current to the motor 304 or to prevent electrical current from flowing to the motor 304. The on/off switch 306 is preferably squeezable, but is it hereby contemplated that the on/off switch 306 may be any other type of switch. The motor 304, the valve 112, 212 and the on/off switch 306 are preferably configured to be a single mechanism, however it is further contemplated that any of, or all of the valve 112, 212, motor 304 and the on/off switch 306 may be standalone and operate as a unit.

Optionally, and or additionally, an adaptor 114, 214 is configured for providing a secure and safe coupling between the pipe 111, 211 and the port 303 of the holding tank 300.

Optionally, and or additionally, the body 101, 201 of the catheters 100, 200 has a power switch 140, 240. The power switch 140, 240 allows easy access to have the holding tank 300 power on without having to bend or reach to turn the holding tank on. The power switch 140, 240 has an electrical wire 141, 241 coupled to a second plug 142, 242. The second plug 142, 242 receives the power source coupler 305 and is coupled to the power source 350. The power switch 140, 240 allows or terminates electrical energy from the power source 350 to the holding tank 300.

In the numbered clauses below, specific combinations of aspects and embodiments are articulated in a shorthand form such that (1) according to respective embodiments, for each instance in which a “component” or other such identifiers appear to be introduced (with “a” or “an,” e.g.) more than once in a given chain of clauses, such designations may either identify the same entity or distinct entities; and (2) what might be called “dependent” clauses below may or may not incorporate, in respective embodiments, the features of “independent” clauses to which they refer or other features described above.

Those skilled in the art will appreciate that the foregoing specific exemplary processes and/or devices and/or technologies are representative of more general processes and/or devices and/or technologies taught elsewhere herein, such as in the claims filed herewith and/or elsewhere in the present application.

The features described with respect to one embodiment may be applied to other embodiments or combined with or interchanged with the features of other embodiments, as appropriate, without departing from the scope of the present invention.

Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. An external catheter for providing medical help to relieve a bladder of a person, the catheter comprising:
a body, the body comprising:
a receiving portion, the receiving portion for receiving liquid waste from a person;
a inside, the inside being accessed through the receiving portion, the inside being for guiding the liquid waste;
a drain, the drain being coupled to an external vacuum holding tank; and
a motor, the motor coupled to a power source, the motor being coupled to the drain tank and thereby increasing a flow of the liquid waste into the holding tank.

2. The catheter of claim 1, wherein the body further comprising: a first switch, the first switch being for allowing flow of the waste product to the drain.

3. The catheter of claim 2, wherein the first switch being a squeeze switch.

4. The catheter of claim 2, wherein the pipe tube further comprising a valve, the valve for allowing a flow of the waste product, wherein the valve being coupled to a motor.

5. The catheter of claim 4, wherein the valve being a solenoid valve.

6. The catheter of claim 5, wherein the valve, the first switch and the motor being in a single mechanism.

7. The catheter of claim 1, wherein further comprising: a pipe tube, the pipe tube being coupled to the drain.

8. The catheter of claim 1, wherein the pipe tube further comprising a valve, the valve for allowing a flow of the waste product, wherein the valve being coupled to a motor.

9. The catheter of claim 8, wherein the valve being a solenoid valve.

10. The catheter of claim 1, wherein the top further comprising: an outer rim.

11. The catheter of claim 10, wherein the outer rim further comprising a ridge.

12. The catheter of claim 11, wherein the ridge having a width being one-eighth (⅛) inch.
